(19) United States
(12) Patent Application Publication
(10) Pub. No.: US 2026/0338905 A1
(43) Pub. Date: Oct. 29, 2026

(54) PHANTOM APPARATUSES AND METHODS THEREFOR

(71) Applicant: Calimetrix LLC, Middleton, WI (US)

(72) Inventors: Jean H. Brittain, Middleton, WI (US); David R. Rutkowski, North Bend, WA (US)

(21) Appl. No.: 19/191,742

(22) Filed: Apr. 28, 2025

(51) Int. Cl. G01R 33/58; A61B 5/055; A61B 6/03; A61B 6/58

(52) U.S. Cl. CPC G01R 33/58; A61B 6/583; A61B 5/055; A61B 6/032

(57) ABSTRACT

Aspects of the disclosure are directed to apparatuses and methods involving a plurality of self-contained tissue-mimicking components and a sealed housing. At least one of the tissue-mimicking components has a tissue property that is different than a tissue property of one of the other tissue-mimicking components. The sealed housing is filled with material and has respective apertures recessed therein. Each aperture has a shape that corresponds to the shape of one of the tissue-mimicking components to receive and hold the one of the tissue-mimicking components in a fixed configuration within the aperture. Certain aspects are directed to utilizing the tissue-mimicking components in magnetic resonance imaging (MRI) or computed tomography (CT), such as to assess the operation of MRJ or CT equipment.

### BACKGROUND

For imaging and diagnostic type equipment, it can be desirable to utilize testing componentry to assess the equipment operation and/or to assess the capability of users of the equipment, for instance as may be used in clinical care, in research, and in clinical trials of new treatments for disease. One such approach involves the utilization of a phantom (test object) designed to be imaged and/or scanned, for testing of equipment and software carrying out the imaging and/or scanning. For instance, phantoms may be used with magnetic resonance imaging (MRI) and computed tomography (CT). Tissue-mimicking phantoms may serve as reference standards for assessing relevant equipment and software, and/or for assessing the manner in which such equipment and software are being used.

As imaging methods are developed and used, quality control (QC) and assurance (QA) are needed to verify that the imaging systems are functioning correctly. Testing and QA/QC using human volunteers can be costly, time-consuming, have low repeatability, and do not provide a reference standard for comparison of results.

While useful, phantoms may be limited in their function, and may need to be tailored to the particular application in which they are utilized. These and other matters have presented challenges to the design and implementation of phantoms for a variety of applications.

### SUMMARY

Various embodiments are directed to phantoms, their application and their manufacture, as may address the issues noted above. A particular embodiment is directed to an apparatus having a housing to receive tissue-mimicking components, in which the housing is sealed and the tissue-mimicking components may be inserted, removed and changed. The housing may have receptacles shaped to correspond to the shape(s) of the tissue mimicking components, for secure holding. Such embodiments may be useful for assessing the efficacy and performance of MRI, CT, or other types of imaging equipment.

As may be implemented in accordance with one or more embodiments, an apparatus includes a plurality of self-contained tissue-mimicking components and a sealed housing. The tissue-mimicking components include at least one tissue-mimicking component having a tissue property that is different than a tissue property of one of the other tissue-mimicking components. The housing is filled with material and has respective apertures recessed therein. Each aperture has a shape that corresponds to the shape of one of the tissue-mimicking components to receive and hold the one of the tissue-mimicking components in a fixed configuration within the aperture.

Another embodiment is directed to a method as follows. A plurality of self-contained tissue-mimicking components are provided, with at least one tissue-mimicking component having a tissue property that is different than a tissue property of one of the other tissue-mimicking components. A sealed housing filled with material is provided, with the housing having respective apertures recessed therein, each aperture having a shape that corresponds to the shape of one of the tissue-mimicking components to receive and hold the one of the tissue-mimicking components in a fixed configuration within the aperture. Respective ones of the plurality of tissue-mimicking components are placed into the respective apertures.

The above discussion/summary is not intended to describe each embodiment or every implementation of the present disclosure. The figures and detailed description that follow also exemplify various embodiments.

### BRIEF DESCRIPTION OF FIGURES

Various example embodiments may be more completely understood in consideration of the following detailed description and in connection with the accompanying drawings, in which:

FIGS. 1A-1C show a phantom apparatus in accordance with one or more embodiments, in which:

FIG. 1A shows tissue-mimicking regions coupled to one another in a linear arrangement,

FIG. 1B shows the tissue-mimicking regions of FIG. 1A in a cylindrical arrangement, and

FIG. 1C shows the tissue-mimicking regions of FIG. 1B coupled to respective housings;

FIGS. 2A-2C show a phantom apparatus in accordance with one or more embodiments, in which:

FIG. 2A shows tissue-mimicking regions, which may be coupled to one another in a linear arrangement,

FIG. 2B shows the tissue-mimicking regions of FIG. 2A in a cylindrical arrangement, and

FIG. 2C shows the tissue-mimicking regions of FIG. 2B with a central connecting rod;

FIGS. 3A-3C show a phantom apparatus in accordance with one or more embodiments, in which:

FIG. 3A shows an oblique view,

FIG. 3B shows a front view, and

FIG. 3C shows a side view;

FIGS. 4A-4C show a phantom apparatus in accordance with one or more embodiments, in which:

FIG. 4A shows an oblique view,

FIG. 4B shows a front view, and

FIG. 4C shows a side view;

FIGS. 5A-5B show a phantom apparatus with a housing, in accordance with one or more embodiments, in which:

FIG. 5A shows the housing in a closed position, and

FIG. 5B shows the housing in an open position;

FIGS. 6A-6B show a phantom apparatus with a housing, in accordance with one or more embodiments, in which:

FIG. 6A shows the housing in a closed position, and

FIG. 6B shows the housing in an open position;

FIGS. 7A-7B show a phantom apparatus with a housing, in accordance with one or more embodiments, in which:

FIG. 7A shows the housing in a closed position, and

FIG. 7B shows the housing in an open position;

FIG. 8 shows a phantom apparatus with a housing, in accordance with one or more embodiments;

FIG. 9 shows a phantom apparatus with a housing, in accordance with one or more embodiments;

FIGS. 10A-10B show a phantom apparatus with a housing, in accordance with one or more embodiments, in which:

FIG. 10A shows the apparatus in an open position, and

FIG. 10B shows the apparatus in a closed position;

FIGS. 11A-11D show a phantom apparatus in accordance with one or more embodiments, in which:

FIG. 11A shows an assembly view,

FIG. 11B shows an assembled view, and

FIG. 11C shows a front view with an example internal configuration, and

FIG. 11D shows a front view with another example internal configuration;

FIG. 12 shows a phantom apparatus with a housing, in accordance with one or more embodiments;

FIG. 13 shows a phantom apparatus with a housing, in accordance with one or more embodiments;

FIG. 14 shows a phantom apparatus with a housing, in accordance with one or more embodiments;

FIG. 15 shows a phantom apparatus with a housing, in accordance with one or more embodiments;

FIG. 16 shows a phantom apparatus with a housing, in accordance with one or more embodiments;

FIG. 17 shows a phantom apparatus with a housing, in accordance with one or more embodiments;

FIG. 18 shows a phantom apparatus with a housing, in accordance with one or more embodiments;

FIG. 19 shows a phantom apparatus with a housing, in accordance with one or more embodiments; and

FIG. 20 shows a phantom apparatus with a housing, in accordance with one or more embodiments.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as may be used throughout this application is by way of illustration, and not limitation.

### DETAILED DESCRIPTION

Aspects of the present disclosure are believed to be applicable to a variety of different types of articles of manufacture, apparatuses, systems and methods involving phantoms. In certain implementations, aspects of the present disclosure have been shown to be beneficial when used in the context of phantoms having tissue-mimicking components and a housing for receiving/holding the tissue-mimicking components. Various embodiments are directed to such phantoms that facilitate high quality quantitative measurements, for instance over a range of human in-vivo tissue having fat concentration or MRI relaxation time constants (T1, T2, R2*, T1rho). While not necessarily so limited, various aspects may be appreciated through a discussion of examples using such exemplary contexts.

Various embodiments are directed to a phantom apparatus having tissue-mimicking components operable in both a housing mode and a pack mode that may omit use of a housing, such as may be utilized in MRI, CT and other cross-sectional imaging modalities. The apparatus may thus be amenable to utilization in an imaging system bore along with an imaging subject (e.g., a patient or volunteer), in the pack mode without requiring the housing (which may be bulky). Further, individual tissue mimicking compartments can be easily replaced or moved, facilitating refurbishment of the pack mode or housing mode. When operated in the pack mode, the phantom (or parts of the phantom) may be placed underneath a subject, for example using an optional pad or other holding/positioning device, strapped to the subject with a belt, or rested on or beside the subject. The pack mode may provide a quality assurance assessment by embedding a phantom reference in the same image as the patient, thus enabling detection of errors in imaging results. Accordingly, when utilizing the housing mode, desirable image quality may be obtained when scanned independently for periodic quality assurance. When utilizing the pack mode, aspects of the phantoms may be scanned at the same time as a subject to provide a reference standard embedded in the subject's image.

By enabling a user to position the compartments (e.g. vials) containing the tissue-mimicking materials into both a housing-based phantom configuration and a pack-type configuration, the cost to users who desire optimal image quality for both applications will be reduced. These aspects will also enable users to replace only the compartments (e.g., vials) containing the tissue-mimicking materials in their phantom without replacing the housing components that have longer life expectancy.

In certain implementations, one or more reference standards are inserted into or onto the phantom housing, enabling simultaneous imaging of original (older) and new (freshly made) phantoms, or phantoms having different tissue mimicking materials. These approaches may facilitate user-based assessment of phantom stability. Further, remote analysis of customer-acquired phantom data may be provided for remote verification of phantom stability.

Generally, as used herein, the term “tissue-mimicking component” refers to a component having characteristics that mimics one or more properties of human tissue. Such components may include one or more of a variety of materials, such as a gel (e.g., water-based gels), plastics, silicones, epoxy, polyurethane foams, carbon powder, agar, agarose, gelatin, polyvinyl alcohol, and various solutions containing chemicals such as nickel chloride, manganese chloride, and sodium chloride. The composition of respective tissue-mimicking components may be varied, for example by including different materials or different concentrations of materials. These materials may be housed in glass, plastic or other containing walls that make up the tissue-mimicking component.

For general information regarding phantoms, and for specific information as to various phantom approaches as may be implemented in accordance with one or more embodiments herein, reference may be made to U.S. patent application Ser. No. 17/959,830 filed on Oct. 4, 2022, and entitled: PHANTOM APPARATUS AND METHODS THEREFOR, and which claims the benefit of U.S. Provisional Application Ser. No. 63/251,908 filed on Oct. 4, 2021, which are fully incorporated herein by reference.

As may be implemented in accordance with one or more embodiments, an apparatus includes a plurality of self-contained tissue-mimicking components and one or more sealed housings having apertures shaped to receive the tissue-mimicking components. The tissue-mimicking components may have different tissue properties relative to each other. The housing may be solid or filled with one or more materials, for example also having tissue-mimicking characteristics. The apertures may be shaped to correspond to the shape of one or more of the tissue-mimicking components, such that it can receive and hold the tissue-mimicking components in a fixed configuration within the aperture. Further, one or more of the tissue-mimicking components may be coupled to an exterior surface of the sealed housing.

The housings used for the tissue-mimicking components as characterized herein may be formed of one or more of a variety of materials. For instance, the housings may be solid, or may be fillable such that the housing can be filled with materials to suit particular applications. For instance, the housing may be filled with a fluid that facilitates magnetic resonance imaging of the apparatus, such as liquid, gels, and gas. The housing may also be filled with solid materials, such as polymers that mimic imaging properties for a given imaging system (e.g. a material with properties close to water for MR imaging).

In a particular embodiment, such a housing has a continuous exterior surface within which material is sealed, and the apertures are defined by respective portions of the continuous exterior surface. Each aperture holds at least a portion of one of the tissue-mimicking components surrounded by the respective portions of the continuous exterior surface and the material sealed within. For instance, an aperture may hold an entire tissue-mimicking component, or a portion thereof in which another portion extends out of the aperture. In a particular embodiment, the housing is an end cap that houses an end of each of one or more of the tissue-mimicking components, with at least one of the tissue-mimicking components extending out of the end cap.

The tissue-mimicking components may be connected by one or more couplers that flexibly connect two or more of the tissue-mimicking components to one another. This approach may facilitate utilization of the connected tissue-mimicking components in a configuration in which the connected tissue-mimicking components are removed from the housing and remain flexibly connected to one another. Flexible couplers may include, for example, fabric couplers, plastic couplers, straps, hook-and-loop fasteners, clip fasteners, and a combination of these and/or other components.

In a particular implementation in which respective ones of the tissue-mimicking components are connected to one another by one or more couplers, the tissue-mimicking components operate in respective configurations including a configuration in which the tissue-mimicking components are removed from the housing, and a configuration to facilitate insertion of the tissue-mimicking components into the respective apertures while the tissue-mimicking components are connected. Such an approach may, for example, facilitate arrangement of the tissue-mimicking components in a linear or other configuration. When removed from the housing, the tissue-mimicking components may be used in connection with imaging of a patient, for example by placement under, alongside, or on the patient. The tissue-mimicking components may be rolled into a cylindrical arrangement for alignment to the apertures and insertion into the housing.

In a particular implementation, the housing has a sealable opening that may be opened and re-sealed, facilitating filling the housing with the material and subsequent sealing of the material within the housing. For instance, a removable fill port plug or a flexibly connected flap or hatch may be fixed to an outer surface of the housing for sealing an opening in the outer surface. Other approaches involve a fill port plug that is detachable (e.g., via a threaded opening) for filling.

Various embodiments include one or more additional sealed housings also having material (solid or filled with material) and respective apertures recessed therein. For instance, a second sealed housing may be used to form a second end cap, relative to the first end cap noted above. The respective end caps may hold opposing portions of each of the tissue-mimicking components. Further, the end caps may be filled with tissue-mimicking components of the same or different materials.

In another embodiment, the second sealed housing forms an enclosure together with the aforementioned housing that encloses all of the tissue-mimicking components. The enclosure may have a continuous exterior surface provided via the respective sealed housings, with the tissue-mimicking components enclosed within the exterior surface.

Another embodiment involving a second sealed housing utilizes both housings to enclose all of the tissue-mimicking components, with at least one of the tissue-mimicking components having respective portions received and held by each of the housings. As such, the respective housings may provide a sealed outer shell that holds tissue-mimicking components therein. In certain implementations, a center rod is placed between the tissue-mimicking components, which wrap around the center rod as they are held by the respective housings.

In a further embodiment involving a second sealed housing, at least one aperture in one of the respective housings holds all of one of the tissue-mimicking components within the at least one aperture. The respective sealed housings forming an enclosure together that enclose all of the tissue-mimicking components.

Another embodiment is directed to a method as follows. Self-contained tissue-mimicking components are provided, including one or more tissue-mimicking components having a tissue property that is different than a tissue property of one of the other tissue-mimicking components. A sealed housing filled with material is provided, in which the housing has respective apertures recessed therein. Each aperture has a shape that corresponds to the shape of one of the tissue-mimicking components, to receive and hold the one of the tissue-mimicking components in a fixed configuration within the aperture. For instance, an aperture may have an interior surface that corresponds to an exterior surface of a tissue-mimicking component, providing a snug fit. Respective ones of the plurality of tissue-mimicking components may be placed into the respective apertures.

The housing may have a continuous exterior surface within which the material is sealed. The apertures are defined by respective portions of the continuous exterior surface and hold at least a portion of one of the tissue-mimicking components surrounded by the respective portions of the continuous exterior surface and the material sealed within.

In a particular embodiment, a reference standard tissue-mimicking component is placed into one of the apertures and imaged along with another one of the tissue-mimicking components. The other tissue-mimicking component is assessed relative to the reference standard tissue-mimicking component, based on the imaging.

In various applications, the tissue-mimicking components are flexibly connected using a coupler. The flexibly connected tissue-mimicking components are placed adjacent to a patient and imaged, for example using MRI, CT, or other imaging method. After imaging, the flexibly connected tissue-mimicking components may be inserted into the sealed housing, which is used to maintain the flexibly connected tissue-mimicking components in a fixed configuration. The flexibly connected tissue-mimicking components are imaged in the fixed configuration, for example using MRI, CT, or other imaging method.

A second sealed housing is provided in certain embodiments, and is also similarly filled with material therein (e.g. the same or different material), with apertures shape that corresponds to the shape of one of the tissue-mimicking components. Respective ones of the tissue-mimicking components are placed into the respective apertures of the second sealed housing. An enclosure may be formed by coupling the sealed housings to one another, to enclose all of the tissue-mimicking components within the enclosure.

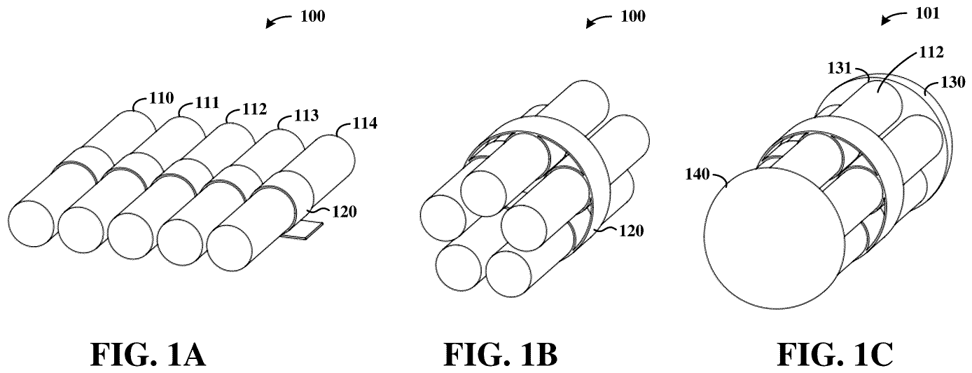

Turning now to the figures, FIGS. 1A-1C show a phantom apparatus 100, and a further assembly apparatus 101, in accordance with one or more embodiments. FIG. 1A shows tissue-mimicking regions 110-114 coupled to one another in a linear arrangement. A coupler 120, such as a strap, holds the respective regions 110-114 together. The tissue-mimicking regions 110-114 may be implemented as a set or strand of vials or other compartments that could be positioned underneath, on-top-of, or on the side of a subject/patient within the imaging bore. The pack could be attached to a patient using a belt or other device, or could simply rest on, beside, or below the patient. In any of these positions, the pack could be in a pad or other type of holder or outside of a pad or other type of holder.

The pack of tissue-mimicking regions 110-114 (e.g. vials) can be rolled or otherwise positioned into a different shape and then fixed within one or more additional objects/structures. In this context, FIG. 1B shows the tissue-mimicking regions of FIG. 1A in a cylindrical arrangement, with the tissue-mimicking regions 110-114 rolled and the coupler 120 is wrapped around as shown.

FIG. 1C shows an assembly apparatus 101, having the apparatus 100 as shown in FIG. 1B coupled to respective housings 130 and 140. The housings (e.g., end caps) 130 and 140 have apertures in which opposing ends of each of the tissue-mimicking regions 110-114 are inserted. For instance, an end of tissue-mimicking region 112 may be inserted into aperture 131. The housings 130 and 140 may “click” fit onto the end of the tissue-mimicking regions. Further, the housings 130 and 140 may be filled with gas, fluid, gel, solid, other material, and may be fabricated from a variety of materials (e.g. polymers) that closely match the desired imaging properties for a given imaging system with which they are to be utilized (e.g. a material with properties close to water for MRI).

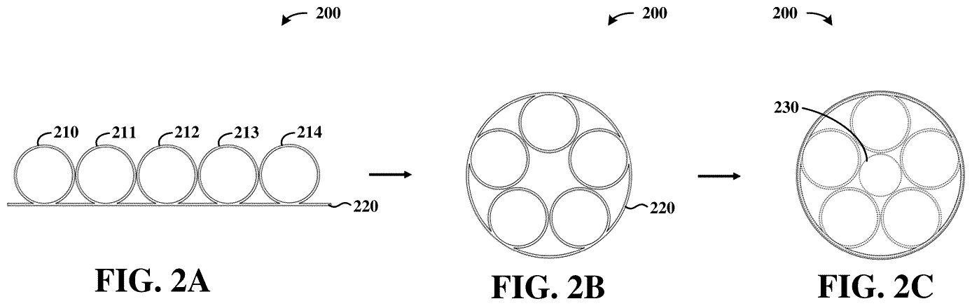

FIGS. 2A-2C show a phantom apparatus 200, as may be implemented in accordance with one or more embodiments. FIG. 2A shows tissue-mimicking regions 210-214, which may be optionally coupled to one another in a linear arrangement. A coupler 220, such as a strap, holds the respective regions 210-214 together. As with the pack in FIGS. 1A-1C, the pack of tissue-mimicking regions 210-214 may be implemented as a set or strand of vials or other compartments that could be positioned underneath, on-top-of, or on the side of a subject/patient within the imaging bore. The pack may be attached to a patient using a belt or other device, or could simply rest on, beside, or below the patient. In any of these positions, the pack could be in a pad or other type of holder or outside of a pad or other type of holder.

FIG. 2B shows the tissue-mimicking regions of FIG. 2A in a cylindrical arrangement with strap 220 wrapped around to maintain the cylindrical arrangement. FIG. 2C shows the tissue-mimicking components of FIG. 2B with a central connecting rod 230 placed within the cylindrical arrangement. As with FIG. 1C, the apparatus 200 may be coupled to respective housings on the opposing ends of the tissue-mimicking components 210-214.

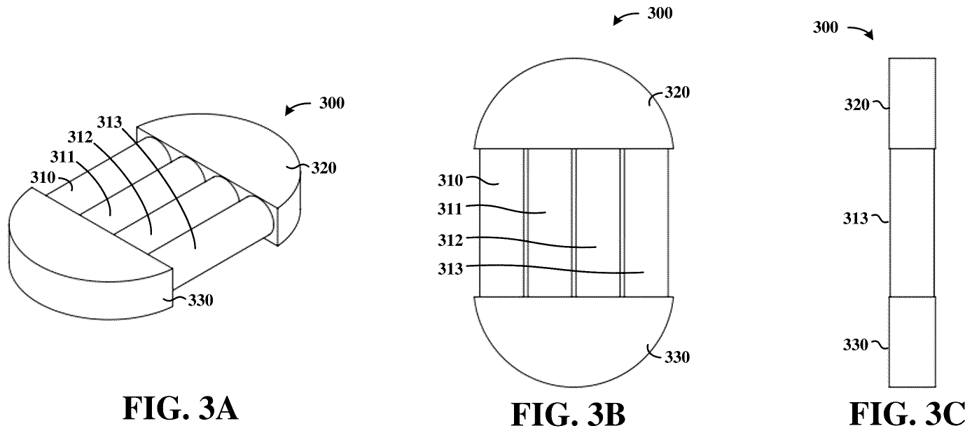

FIGS. 3A-3C show a phantom apparatus 300 in accordance with one or more embodiments, in which FIG. 3A shows an oblique view, FIG. 3B shows a front view, and FIG. 3C shows a side view. The apparatus 300 includes tissue-mimicking regions 310-313 and two rounded end cap housings 320 and 330.

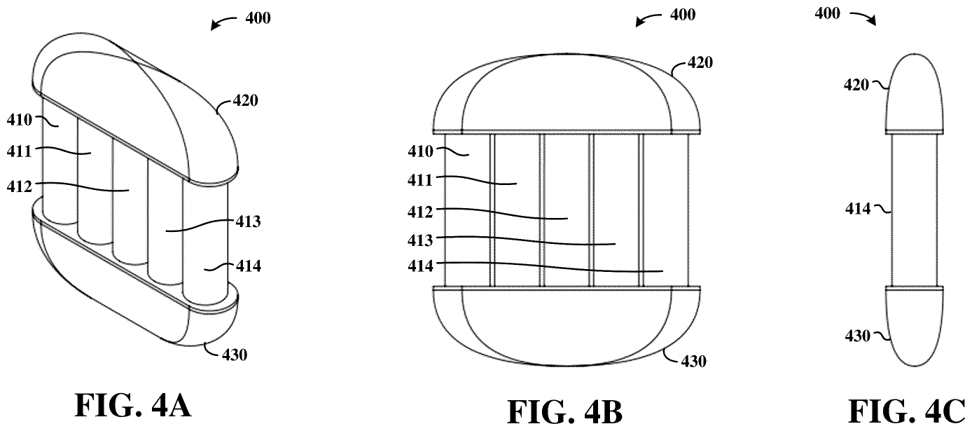

FIGS. 4A-4C show a phantom apparatus 400 in accordance with one or more embodiments, in which FIG. 4A shows an oblique view, FIG. 4B shows a front view, and FIG. 4C shows a side view. The apparatus 400 includes tissue-mimicking regions 410-414 and two end cap housings 420 and 430.

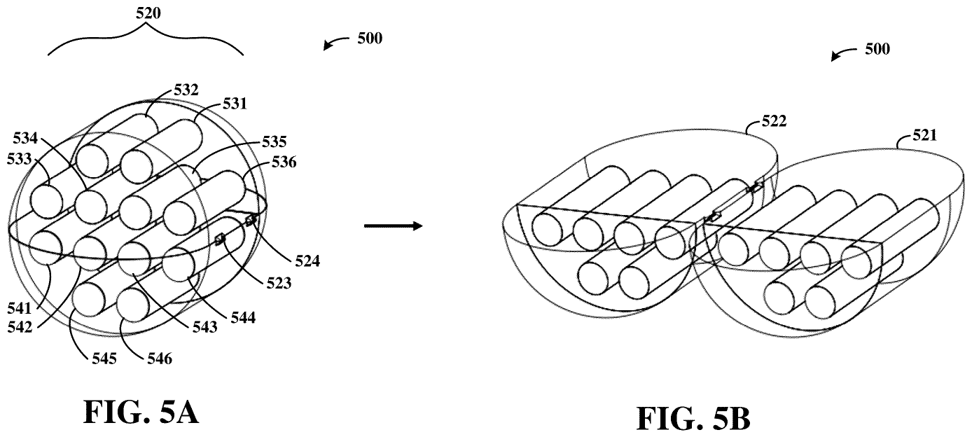

Various embodiments are directed to sectioned housings, such as foldable and/or lay-flat housings, which may be implemented in a variety of manners. Such housings may be pill-shaped, spherical, cylindrical or have other shapes. Each section may be connected by hinges or other swivel-type connections. Such housings may be used in a closed formation on their own, or as an embedded reference standard by laying the housing flat under, on, or beside an imaging subject. FIGS. 5A-7B depict embodiments that may be implemented in this regard. Such housings may further be surrounded by or embedded in pads or another type of holder/structure when used in its “flat” configuration, may be rigid or flexible, and may be filled with gas, liquid, gel, solids, or other tissue-mimicking material.

Beginning with FIGS. 5A-5B, a phantom apparatus 500 has a housing 520 as may be implemented in accordance with one or more embodiments. FIG. 5A shows the housing in a closed position and FIG. 5B shows the housing in an open position. The housing 520 includes upper portion 521 and lower portion 522 connected by hinges 523 and 524. The upper portion 521 houses tissue-mimicking components 531-536, and the lower portion 522 houses tissue-mimicking components 541-546.

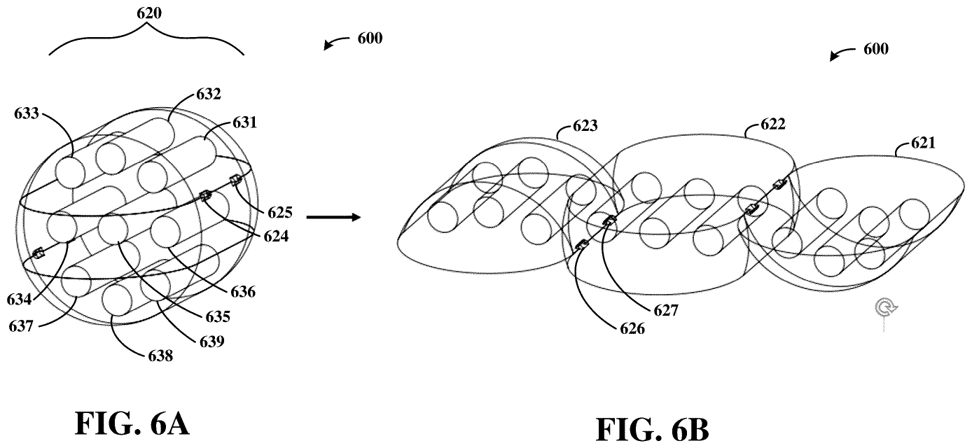

FIGS. 6A-6B show a phantom apparatus 600 with a housing 620 in accordance with one or more embodiments, in which FIG. 6A shows the housing in a closed position and FIG. 6B shows the housing in an open position. The housing 620 has upper portion 621, middle portion 622 and lower portion 623. The upper portion 621 is connected to the middle portion 622 by hinges 624 and 625. The lower portion 623 is connected to the middle portion 622 by hinges 626 and 627. The apparatus 600 also includes a plurality of tissue-mimicking components 631-639, three of which are in each housing portion as shown.

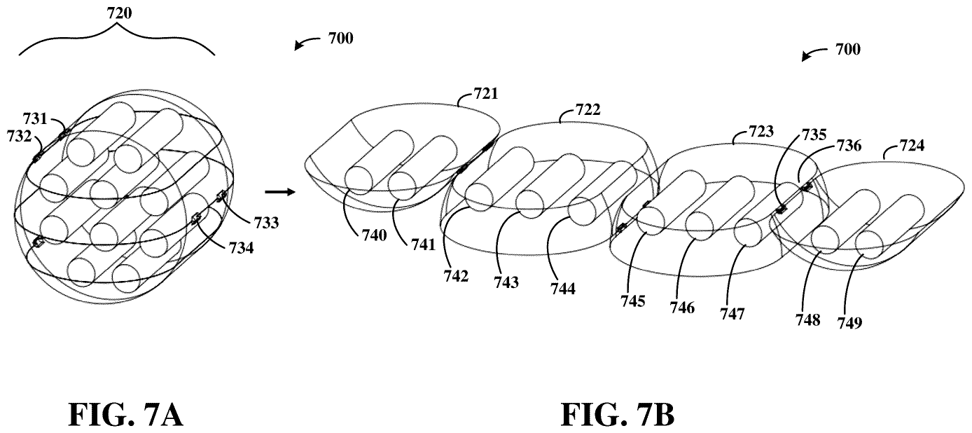

FIGS. 7A-7B show a phantom apparatus 700 with a housing 720 in accordance with one or more embodiments, in which FIG. 7A shows the housing in a closed position and FIG. 7B shows the housing in an open position. The housing 720 has upper portion 721, upper middle portion 722, lower middle portion 723 and lower portion 724. The upper portion 721 is connected to the upper middle portion 722 by hinges 731 and 732. The upper middle portion 722 is connected to the lower middle portion 723 by hinges 733 and 734. The lower middle portion 723 is connected to the lower portion 724 by hinges 735 and 736.

The apparatus 700 also includes a plurality of tissue-mimicking components 740-749. For these tissue-mimicking components, the upper portion 721 houses components 740-741, the upper middle portion 722 houses components 742-744, the lower middle portion 723 houses components 745-747, and the lower portion 724 houses components 748 and 749.

Various embodiments are directed to a housing with externally replaceable vials of tissue-mimicking components. The housing may be modular such that the vials can be easily replaced by a user. For instance, a cylindrical or flat-shaped housing may be filled with fluid, gel, or other material, and includes apertures (e.g., slots) that can hold the vials. The housing may have removable end caps that allow for access to the vials while maintaining desirable image quality. Certain embodiments are directed to vials with end caps that lock together, with the end caps being removable for vial replacement. Further, such end caps could be filled with material to facilitate image quality and could also function as a separate tissue-mimicking compartment. Materials that generate imaging signals or achieve other desirable imaging properties may be placed between the vials.

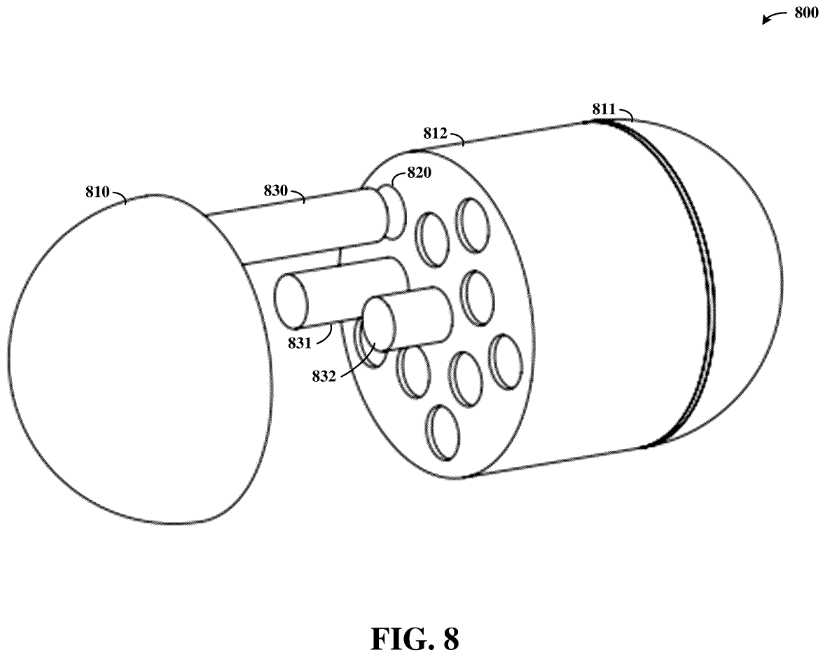

Beginning with FIG. 8, a phantom apparatus 800 is shown in an assembly view, as may be implemented in accordance with one or more embodiments. The apparatus 800 includes a housing having end portions 810 and 811 as well as a middle portion 812. The housing has several apertures (820 labeled by way of example) for receiving tissue-mimicking components. By way of example, tissue-mimicking vials 830, 831 and 832 are shown. Fewer or more vials can be used with the respective apertures as shown, and may be interchangeably replaced.

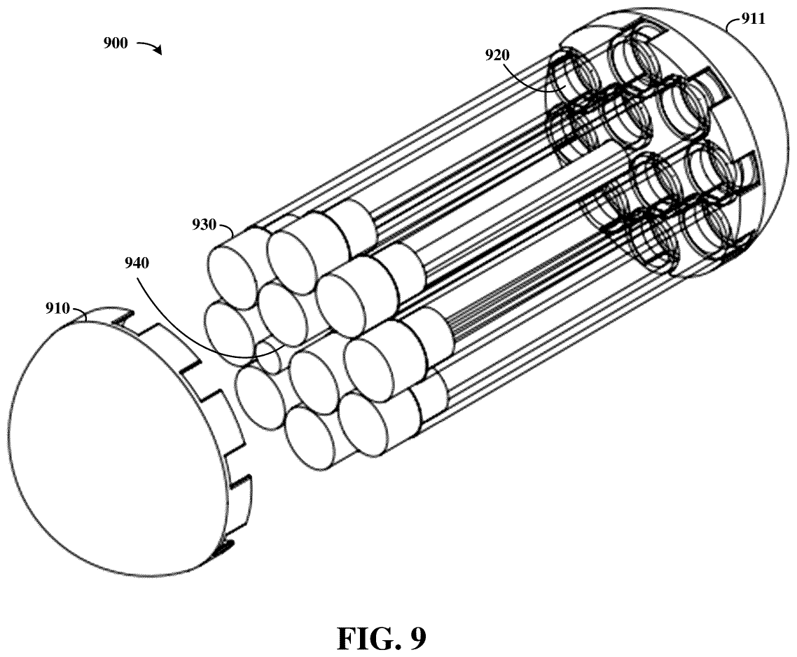

FIG. 9 shows a phantom apparatus 900 with a housing, in accordance with one or more embodiments. The housing of apparatus 900 includes end caps 910 and 911, each having a plurality of apertures for receiving tissue-mimicking components. Aperture 920 and tissue-mimicking component 930 are labeled by way of example, with the aperture 920 configured for holding an end of component 930. A similar aperture in end cap 910 holds an opposing end of component 930. Connecting rod 940 holds the phantom apparatus 900 together. Certain aspects are directed to utilization with small-diameter bore (e.g., preclinical) imaging, where a full-thickness housing may not fit in addition to the vials.

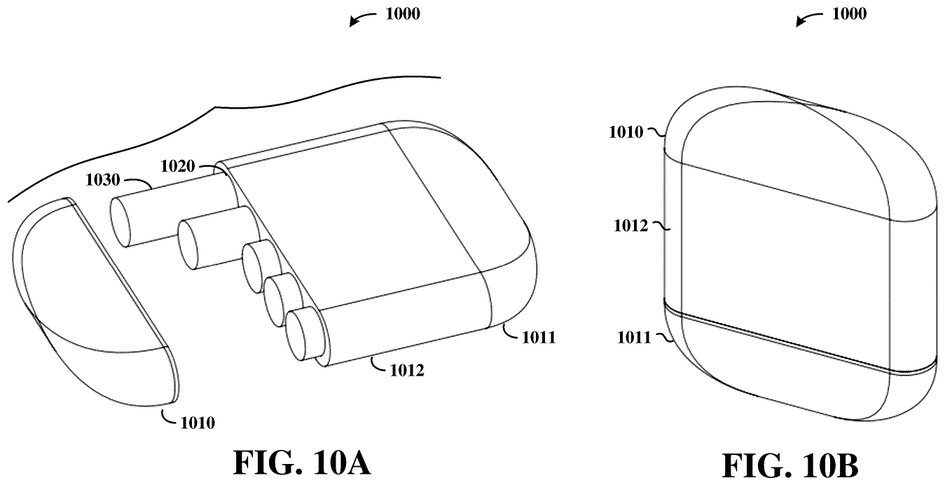

FIGS. 10A-10B show a phantom apparatus 1000 with a housing in accordance with one or more embodiments, in which FIG. 10A shows the apparatus in an open position and FIG. 10B shows the apparatus in a closed position. The housing has end caps 1010 and 1011 as well as a central region 1012, each having a plurality of apertures for receiving and holding tissue-mimicking components. By way of example, aperture 1020 and tissue-mimicking component 1030 are labeled.

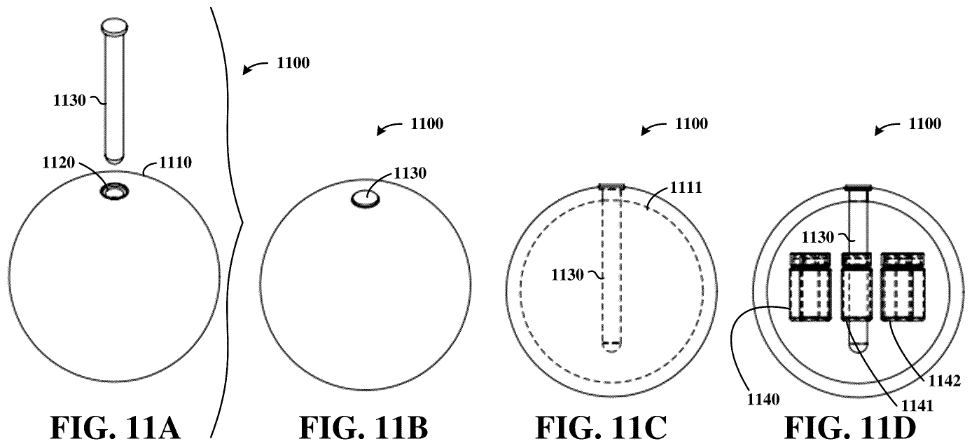

Various embodiments are directed to modular reference standard units that can be appended to or inserted into a phantom, for example to assess longitudinal stability of the phantom. The modular units may be new or “fresh” or of a different material with a longer shelf life, allowing the phantom user to assess the integrity of internal phantom components over time by comparing phantom tissue mimicking component performance to the modular reference standard. One implementation involves using a single compartment (e.g. vial), or a subset of compartments, that can be replaced. Another implementation involves using a replaceable vial that may be inserted through existing functional features of a phantom, such as a fill port. The replaceable vial can be used for quality assurance of the phantom by providing an interchangeable and/or temporary embedded reference standard. Another implementation involves adding reference compartments (e.g. vials) to the outside of a phantom by an embedded feature, a strap, or other device/approach that accomplishes desired positioning. External vials may be used for quality assurance of the phantom periodically, while keeping the internal vials permanent. This allows for longitudinal monitoring of the permanent vials without shipping the phantom back to the manufacturer or dismantling the phantom. FIGS. 11A-14 depict embodiments that may be implemented in this regard.

Beginning with FIGS. 11A-11D, a phantom apparatus 1100 is shown as may be implemented in accordance with one or more embodiments. FIG. 11A shows an assembly view, FIG. 11B shows an assembled view, FIG. 11C shows a front view with an example internal configuration, and FIG. 11D shows a front view with another example internal configuration. The apparatus 1100 has a housing 1110 and an aperture 1120, and a tissue-mimicking component 1130. The aperture 1120 may be a port used for filling the housing with material.

Referring to FIG. 11C, hidden lines are utilized to show an interior sidewall 1111 of the housing 1110, as well as the tissue-mimicking component 1130 inside of the housing. As shown in FIG. 1D, a cross-section of the apparatus 1100 is shown with permanent vials 1140, 1141 and 1142 therein, along with the (replaceable) tissue-mimicking component 1130.

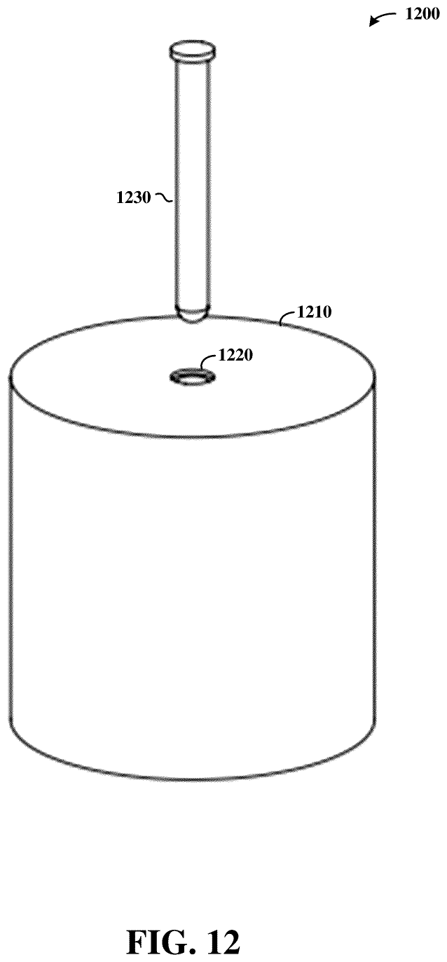

FIG. 12 shows a phantom apparatus 1200, as may be implemented in accordance with one or more embodiments. The apparatus 1200 includes a housing 1210 having an aperture 1220, and a tissue-mimicking component 1230 for insertion into the aperture. The apparatus 1200 may be implemented in a manner similar to that shown with apparatus 1100 in FIG. 11, with additional vials (e.g., permanent) within the housing 1210.

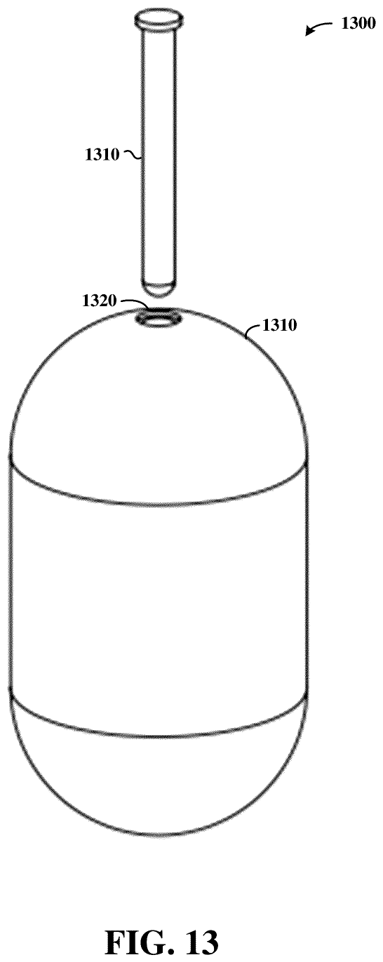

FIG. 13 shows a phantom apparatus 1300, as may be implemented in accordance with one or more embodiments. The apparatus 1300 includes a housing 1310 having an aperture 1320, and a tissue-mimicking component 1330 for insertion into the aperture. The apparatus 1300 may be implemented in a manner similar to that shown with apparatus 1100 in FIG. 11, with additional vials (e.g., permanent) within the housing 1310.

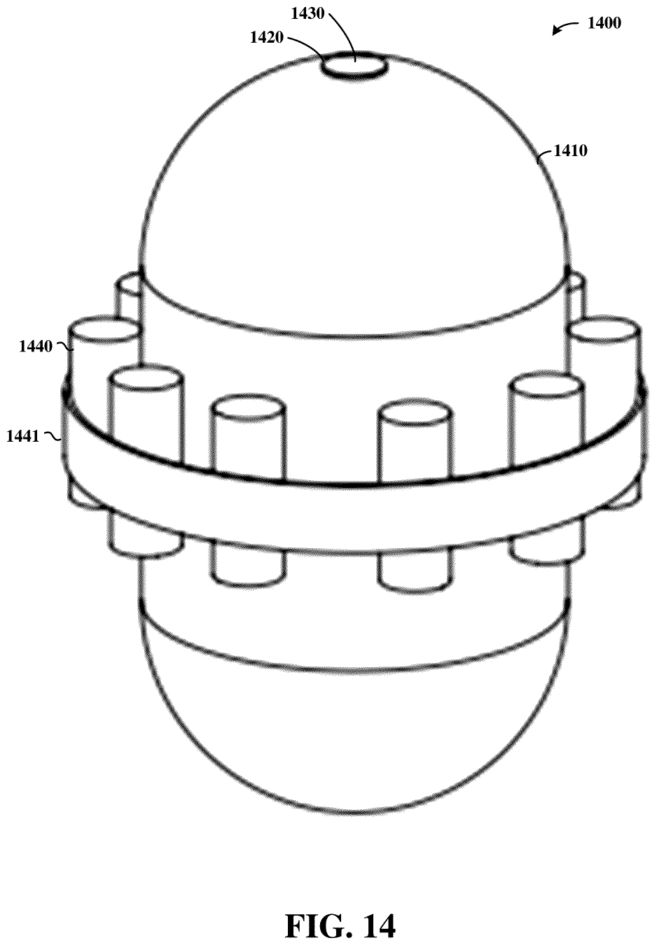

FIG. 14 shows a phantom apparatus 1400, as may be implemented in accordance with one or more embodiments. The apparatus 1400 includes a housing 1410, and may include an aperture 1420 with a tissue-mimicking component 1430 inserted therein (e.g., similar to the apparatus 1300 of FIG. 13). Component 1430 may be a fill-port plug that is removable from aperture 1420 for filling the housing 1410, or such a fill-port plug that is also a tissue-mimicking component. Further, the apparatus 1400 may have a plurality of tissue-mimicking components (e.g., vials, including vial 1440) coupled around the outside of the housing 1410 using a strap 1441. The apparatus 1400 may also be implemented in a manner similar to that shown with apparatus 1100 in FIG. 11, with additional vials (e.g., permanent) within the housing 1410.

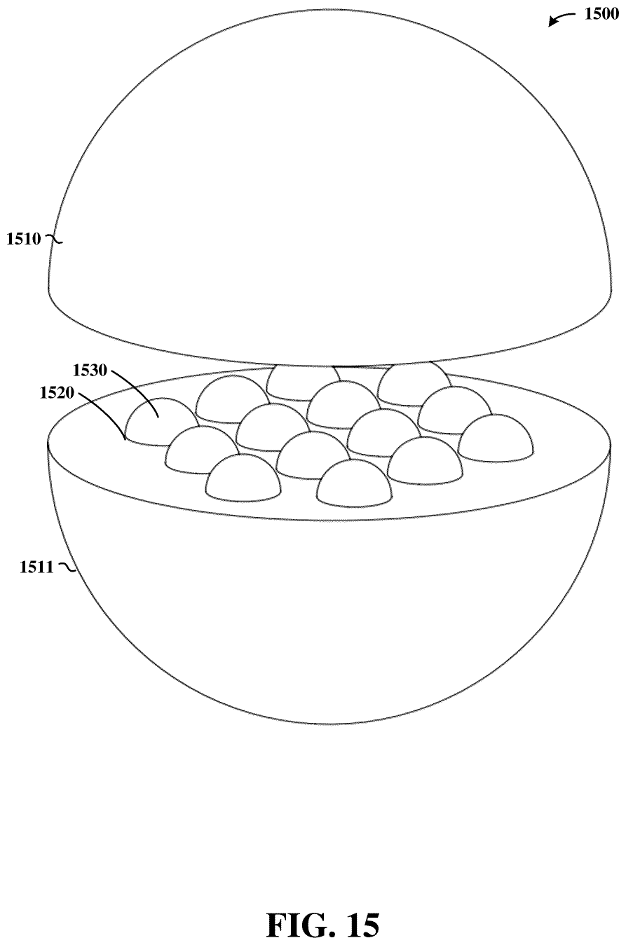

FIGS. 15-20 show a variety of phantom apparatuses in respective shapes and arrangements, as may be implemented in connection with various embodiments. Beginning with FIG. 15, a phantom apparatus 1500 is shown in an exploded view, with upper and lower housing components 1510 and 1511. A plurality of apertures are shown with tissue-mimicking components therein, with aperture 1520 and tissue-mimicking component 1530 labeled by way of example. The tissue-mimicking components may be generally spherical in nature.

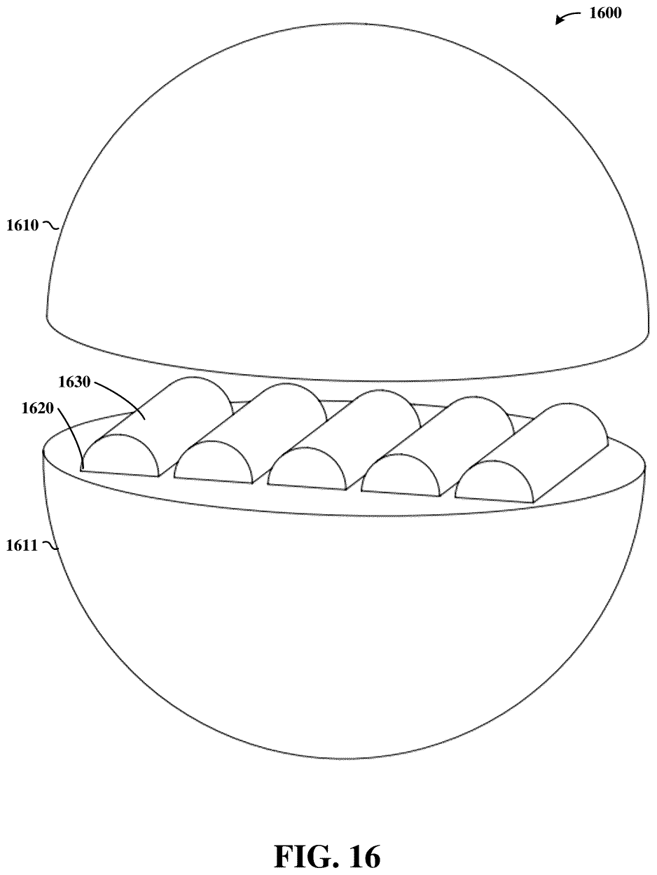

FIG. 16 shows a phantom apparatus 1600 in an exploded view, with upper and lower housing components 1610 and 1611. A plurality of apertures are shown with tissue-mimicking components therein, with aperture 1620 and tissue-mimicking component 1630 labeled by way of example.

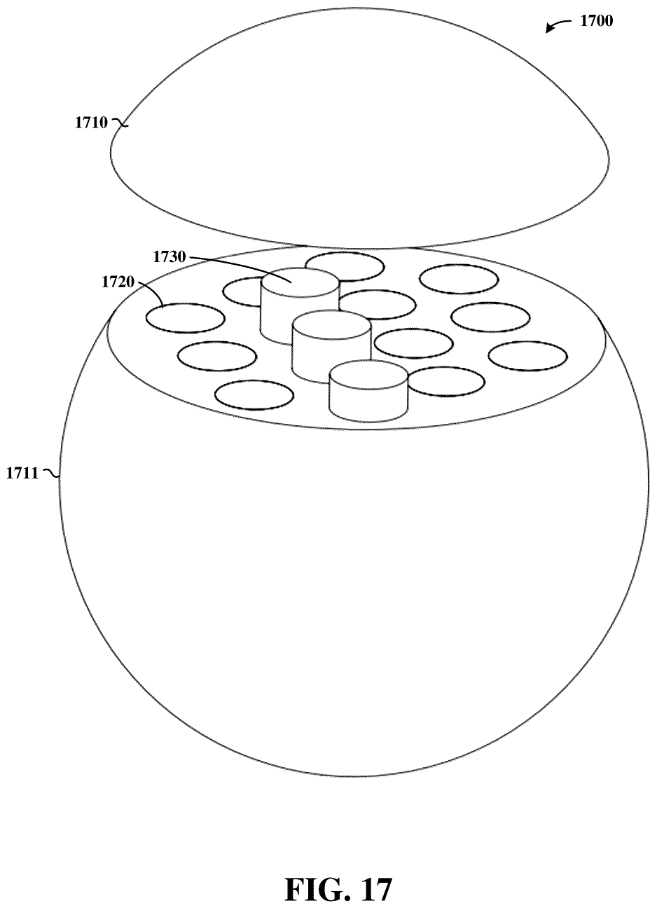

FIG. 17 shows a phantom apparatus 1700 in an exploded view, with upper and lower housing components 1710 and 1711. A plurality of apertures are shown with tissue-mimicking components therein, with aperture 1720 and tissue-mimicking component 1730 labeled by way of example.

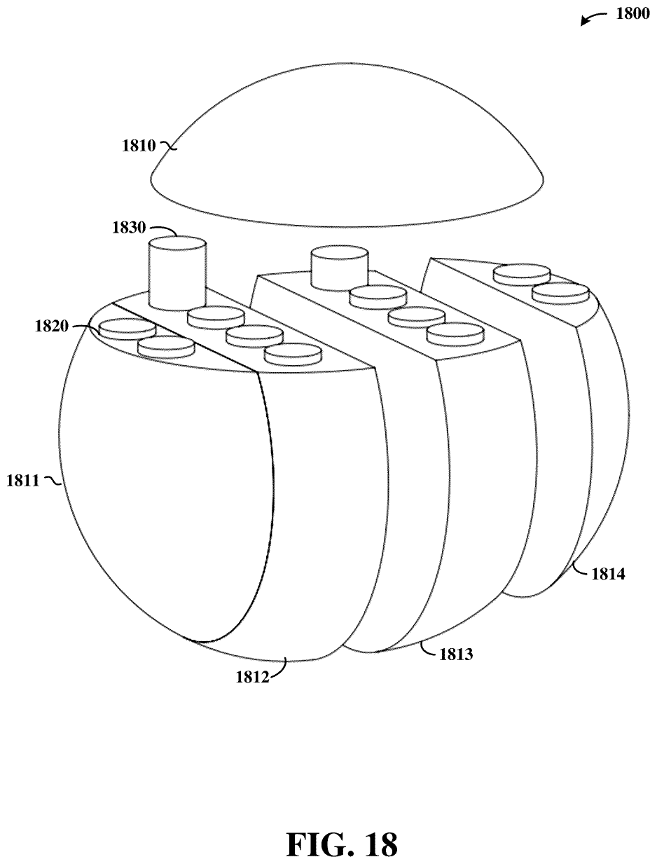

FIG. 18 shows a phantom apparatus 1800 in an exploded view, with upper housing component 1810 and lower housing components including sections 1811-1814. A plurality of apertures are shown with tissue-mimicking components therein, with aperture 1820 and tissue-mimicking component 1830 labeled by way of example.

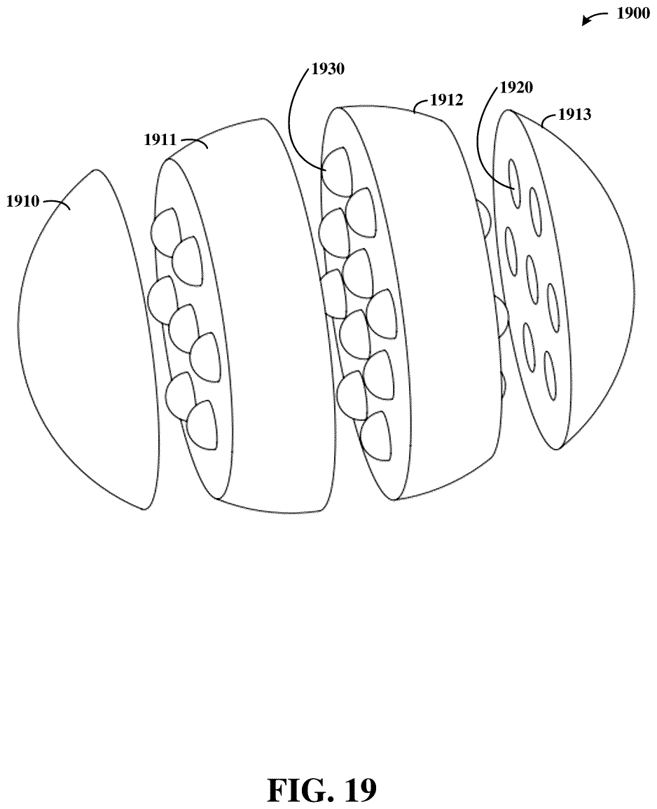

FIG. 19 shows a phantom apparatus 1900 in an exploded view, with housing components 1910, 1911, 1912 and 1913. A plurality of apertures are shown, some of which have tissue-mimicking components therein. Aperture 1920 and tissue-mimicking component 1930 are labeled by way of example.

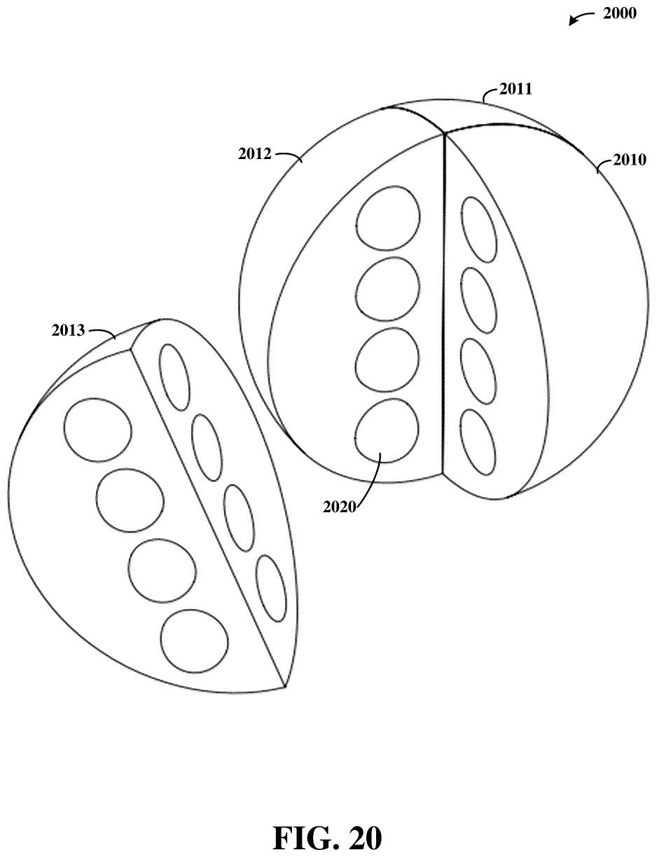

FIG. 20 shows a phantom apparatus 2000 in an exploded view, with housing components 2010, 2011, 2012 and 2013. A plurality of apertures are shown, with aperture 2020 labeled by way of example. Each aperture may hold a tissue-mimicking component, for example as may be held in part by adjacent housing components.

Based upon the above discussion and illustrations, those skilled in the art will readily recognize that various modifications and changes may be made to the various embodiments without strictly following the exemplary embodiments and applications illustrated and described herein. For example, a variety of different shapes and sizes of housings, apertures and tissue-mimicking components may be utilized. Further, different aspects shown separately may be utilized together, such as by utilizing replaceable tissue-mimicking components in FIGS. 5-7. Such modifications do not depart from the true spirit and scope of various aspects of the invention, including aspects set forth in the claims.

What is claimed is:

1. An apparatus comprising:
a plurality of self-contained tissue-mimicking components, including at least one tissue-mimicking component having a tissue property that is different than a tissue property of one of the other tissue-mimicking components; and
a sealed housing filled with material, the housing having respective apertures recessed therein, each aperture having a shape that corresponds to the shape of one of the tissue-mimicking components to receive and hold the one of the tissue-mimicking components in a fixed configuration within the aperture.

2. The apparatus of claim 1, wherein:
the housing has a continuous exterior surface within which the material is sealed; and
the apertures are defined by respective portions of the continuous exterior surface and are configured to hold at least a portion of one of the tissue-mimicking components surrounded by the respective portions of the continuous exterior surface and the material sealed within.

3. The apparatus of claim 1, wherein the sealed housing is an end cap configured to house an end of each of the tissue-mimicking components with at least one of the tissue-mimicking components extending out of the end cap.

4. The apparatus of claim 3, further including a second sealed housing forming a second end cap, the second sealed housing also being filled with material and having respective apertures recessed therein, each aperture having a shape that corresponds to the shape of one of the tissue-mimicking components to receive and hold the one of the tissue-mimicking components in a fixed configuration within the aperture.

5. The apparatus of claim 4, wherein the end cap and the second end cap are respectively configured to hold opposing portions of each of the tissue-mimicking components.

6. The apparatus of claim 1, further including a second sealed housing filled with material and having respective apertures recessed therein, each aperture having a shape that corresponds to the shape of one of the tissue-mimicking components to receive and hold the one of the tissue-mimicking components in a fixed configuration within the aperture, the respective sealed housings forming an enclosure together that enclose all of the tissue-mimicking components.

7. The apparatus of claim 6, wherein the enclosure has a continuous exterior surface provided via the respective sealed housings, with the tissue-mimicking components enclosed within the exterior surface.

8. The apparatus of claim 1, further including a second sealed housing filled with material and having respective apertures recessed therein, each aperture having a shape that corresponds to the shape of one of the tissue-mimicking components to receive and hold the one of the tissue-mimicking components in a fixed configuration within the aperture, the respective sealed housings forming an enclosure together that enclose all of the tissue-mimicking components with at least one of the tissue-mimicking components having respective portions received and held by each of the housings.

9. The apparatus of claim 1, further including a second sealed housing filled with material and having respective apertures recessed therein, each aperture having a shape that corresponds to the shape of one of the tissue-mimicking components to receive and hold the one of the tissue-mimicking components in a fixed configuration, at least one aperture in one of the respective housings being configured to hold all of one of the tissue-mimicking components within the at least one aperture, the respective sealed housings forming an enclosure together that enclose all of the tissue-mimicking components.

10. The apparatus of claim 1, further including at least one coupler that flexibly connects at least two of the tissue-mimicking components to one another, facilitating utilization of the connected tissue-mimicking components in a configuration in which the connected tissue-mimicking components are removed from the housing and remain flexibly connected to one another.

11. The apparatus of claim 1, wherein:
respective ones of the tissue-mimicking components are connected to one another via at least one coupler; and
the tissue-mimicking components are configured and arranged with the at least one coupler to facilitate arrangement of the tissue-mimicking components in a configuration in which the tissue-mimicking components are removed from the housing, and to facilitate insertion of the tissue-mimicking components into the respective apertures while the tissue-mimicking components are connected by the at least one coupler.

12. The apparatus of claim 1, wherein the material includes a fluid material that facilitates magnetic resonance imaging of the apparatus.

13. The apparatus of claim 1, wherein the sealed housing has a sealable opening that may be opened and re-sealed, facilitating filling the housing with the material and subsequent sealing of the material within the housing.

14. The apparatus of claim 1, wherein at least one of the tissue-mimicking components is coupled to an exterior surface of the sealed housing.

15. A method comprising:
providing a plurality of self-contained tissue-mimicking components, including at least one tissue-mimicking component having a tissue property that is different than a tissue property of one of the other tissue-mimicking components;
providing a sealed housing filled with material, the housing having respective apertures recessed therein, each aperture having a shape that corresponds to the shape of one of the tissue-mimicking components to receive and hold the one of the tissue-mimicking components in a fixed configuration within the aperture; and
placing respective ones of the plurality of tissue-mimicking components into the respective apertures.

16. The method of claim 15, wherein:
the housing has a continuous exterior surface within which the material is sealed; and
the apertures are defined by respective portions of the continuous exterior surface and are configured to hold at least a portion of one of the tissue-mimicking components surrounded by the respective portions of the continuous exterior surface and the material sealed within.

17. The method of claim 15, further including:
providing a second sealed housing filled with material and having respective apertures recessed therein, each aperture having a shape that corresponds to the shape of one of the tissue-mimicking components to receive and hold the one of the tissue-mimicking components in a fixed configuration within the aperture; and
placing respective ones of the plurality of tissue-mimicking components into the respective apertures of the second sealed housing.

18. The method of claim 17, further including forming an enclosure by coupling the housings to one another, to enclose all of the tissue-mimicking components within the enclosure.

19. The method of claim 15, wherein placing respective ones of the plurality of tissue-mimicking components into the respective apertures includes placing a reference standard tissue-mimicking component into one of the apertures, further including:
imaging the reference standard tissue-mimicking component and a second one of the tissue-mimicking components; and
assessing the second one of the tissue-mimicking components relative to the reference standard tissue-mimicking component based on the imaging.

20. The method of claim 15, wherein the tissue-mimicking components are flexibly connected by a coupler, further including:
placing the flexibly connected tissue-mimicking components adjacent to a patient;
imaging the flexibly connected tissue-mimicking components and patient;
after imaging, inserting the flexibly connected tissue-mimicking components into the sealed housing, using the sealed housing to maintain the flexibly connected tissue-mimicking components in a fixed configuration; and
re-imaging the flexibly connected tissue-mimicking components in the fixed configuration.
